(19) United States
(12) Patent Application Publication
(10) Pub. No.: US 2020/0055199 A1
(43) Pub. Date: Feb. 20, 2020

(54) SAMPLE MANIPULATOR

(71) Applicant: BENDFLEX RESEARCH AND DEVELOPMENT PRIVATE LIMITED, Bangalore, Karnataka (IN)

(72) Inventors: Ramnath TJ BABU, Bangalore, Karnataka (IN); Santosh DB BHARGAV, Bangalore, Karnataka (IN)

(21) Appl. No.: 16/343,002

(22) PCT Filed: Aug. 8, 2017
(86) PCT No.: PCT/IN2017/050326

§ 371 Date: Apr. 18, 2019

(30) Foreign Application Priority Data
Oct. 18, 2016 (IN) 201641035522

(51) Int. Cl. B25J 15/12; B25J 7/00; A61B 10/02; B25J 19/02

(52) U.S. Cl. CPC B25J 15/12; B25J 19/02; A61B 10/02; B25J 7/00

(57) ABSTRACT

The invention provides a sample manipulator. The sample manipulator includes a partially resilient frame having a mount for removably positioning the manipulator. A pair of opposingly positioned arms is formed at one end of the frame. A means for actuation of the frame is provided on the frame. The means for actuation facilitates manipulation of the sample.

### FIELD OF INVENTION

The invention generally relates to the field of biomedical engineering and particularly to an apparatus for manipulation of sample.

### BACKGROUND

Holding of a sample and subsequent manipulation, by isolating the sample from a given environment has numerous medical and scientific applications. Examples of manipulation include a task that includes but is not limited to squeezing, gripping, grasping, rolling, stretching, bending, piercing, probing and tearing. Examples of samples include but are not limited to biological cells, tissues, scaffolds, tissue-scaffolds, biopsies, zygotes, organelles, platelets, sand-particles, amorphous, crystals and crystalline structures. Examples of applications include but are not limited to study of single cell interactions, estimation of mechanical properties, cell modification and macromolecular interaction, positioning of samples and probing of samples. Various devices and mechanisms have been designed for gripping samples.

One such device has a pair of fingers which are caused to move in parallel paths or in arcuate paths. A significant disadvantage of the device is loss of tactile sensitivity due to static friction. Another apparatus known is based on a tweezers type mechanism. A significant disadvantage of the apparatus is requirement of long arms to provide near parallel movement of the tips. Further, precise control of the long arms and tips can prove difficult. Yet another technique known to exist in the art discloses a micromanipulation apparatus of micro-sized samples, specifically to selectively hold the micro sized samples. The apparatus includes a micromanipulation device in the form of a hollow frame having a base, resiliently deflectable arms projecting from the base and defining a gap for receiving a micro-sized sample, and a saddle connected to the tips of the arms between the base and the tips. A force generating device applies a force to the saddle to deflect the tips inwards to close the gap about the micro-sized sample. A fixture is provided for supporting the micromanipulation device and force generating device. The force generating device can be a micrometer. One disadvantage of the device is that the need for the saddle brings in complication in the operation of the device. Further, the purpose of the saddle is specifically to bring the deflectable arms together for holding a sample.

The above mentioned apparatuses predominantly suffer from the disadvantage that the apparatuses include plurality of parts joined together. Hence, there is a need for an apparatus that is simple to arrange and use.

### BRIEF DESCRIPTION OF DRAWINGS

So that the manner in which the recited features of the invention can be understood in detail, some of the embodiments are illustrated in the appended drawings. It is to be noted, however, that the appended drawings illustrate only typical embodiments of this invention and are therefore not to be considered limiting of its scope, for the invention may admit to other equally effective embodiments.

FIG. 1 shows a sample manipulator, according to one embodiment of the invention.

FIG. 2 shows a sample manipulator, according to another embodiment of the invention.

FIG. 3 shows a sample manipulator, according to yet another embodiment of the invention.

FIG. 4(a)-4(c) shows different configurations of a pair of arms of the sample manipulator, according to an embodiment of the invention.

FIG. 5(a)-5(g) shows various projections that can be configured, according to various embodiments of the invention.

FIG. 6 shows a perspective view of the sample manipulator, according to an embodiment of the invention.

### SUMMARY OF THE INVENTION

One aspect of the invention provides a sample manipulator. The manipulator includes a partially resilient frame having a mount for removably positioning the manipulator. A pair of opposingly positioned arms is formed at one end of the frame. A means for actuation of the frame is provided on the frame. The means for actuation facilitates manipulation of the sample.

Another aspect of the invention provides a sample manipulator for out of plane manipulation. The manipulator includes a partially resilient frame having at least two locations on the frame for removably positioning the manipulator. At least one pair of opposingly positioned projections is formed at one end of the frame. A means for actuation of the frame is provided on the frame for enabling a multi-axial actuation.

Yet another aspect of the invention provides a sample manipulator that includes a frame having a first end configured for removably fixing the manipulator. An intermediate region is configured to receive an operating means. A second end is configured for releasably holding the sample.

A further aspect of the invention provides a sample manipulator that includes a partially resilient frame. At least two locations are provided on the frame for removably positioning the manipulator. A pair of opposingly positioned arms is formed at one end of the frame. At least one means for actuation of the frame is provided on the frame.

### DETAILED DESCRIPTION OF THE INVENTION

Various embodiments of the invention provide a sample manipulator. The manipulator is based on a class of mechanisms generally referred to as compliant mechanisms. Compliant mechanisms transfer and/or transform motion, force, or energy due to the deformation of an elastic member. Compliant mechanisms are, in general, a partially resilient frame capable of undergoing a temporary deformation to achieve a certain task.

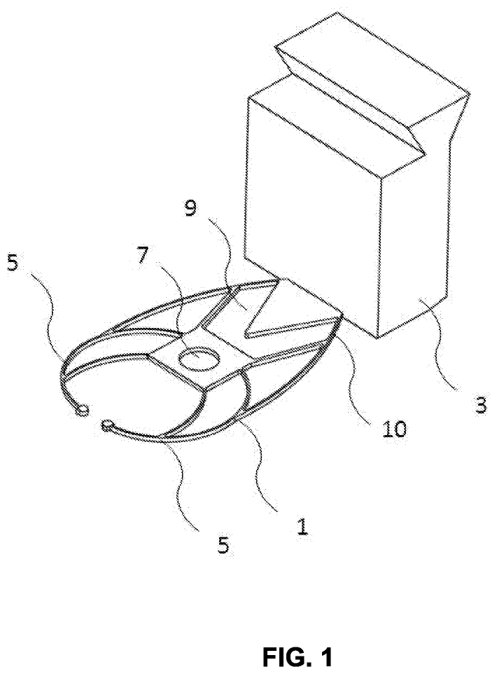

FIG. 1 shows a sample manipulator, according to one embodiment of the invention. The manipulator includes a partially resilient frame 1 having a mount 3 for removably positioning the manipulator. A pair of opposingly positioned arms 5 is formed at one end of the frame 1. A means for actuation 7 of the frame 1 is provided on the frame 1. The means for actuation facilitates manipulation of the sample. The resilience in the frame is due to the choice of the material, the geometry of the frame, or a combination of both. The frame is capable of recovering back from a temporary deformation. Examples of materials include but are not limited to polymers, plastic, composites, metals, Silicon wafer, SU-8 and Silicon on insulator, 3D-printing materials or a combination thereof.

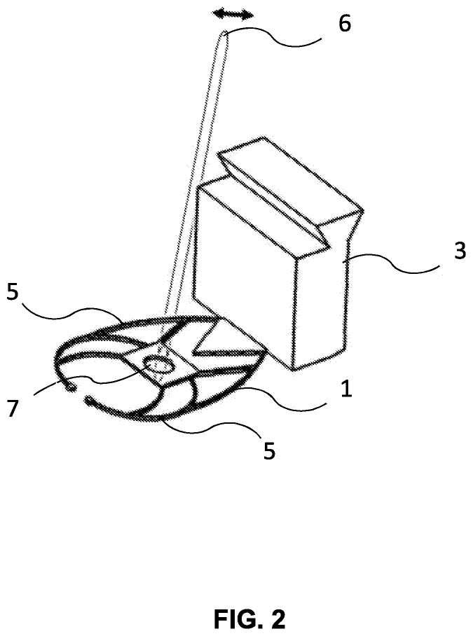

In one example of the invention, the frame along with the pair of arms and the mount is formed out of a single mold. Alternatively, the mount can be affixed to the frame 1, at a predefined location on the frame 1. Examples of affixation include but are not limited to press-fit, snap-fit, fusion, suction-cups, vacuum or gluing. Examples of manufacturing of frame include but are not limited to microfabrcation, electro-discharge machining (EDM), micro-machining, lithography, nanofabrication, surface micromachining, 3D-prinitng, molding, casting, stamping, sheet-metal forming, LASER-cutting, Milling, stencil-cutter, etching, adhesive bonding or a combination thereof. The positioning of the mount 3 depends on the position of means for actuation 7. The means for actuation 7, according to one example of the invention, is a uni-directional actuator. Alternatively, the actuator can be a bi-directional actuator 6 as shown in FIG. 2. Examples of actuator include but are not limited to manual screw based actuator, micrometer screw-gauge, pneumatic actuator, linear actuator, hydraulic actuator, piezo-electric actuator, piezo-resistive actuator, thermal actuator, electro-thermal actuator, electrostatic actuator, Shape memory alloys-based actuator, biohybrid actuator, muscle-based actuator, jet-based or a combination thereof. The mode of actuation includes but are not limited to mechanical, electrical, electronic, force-field based actuation. Further, the mode of actuation can be regulated either at the point of actuation or remotely.

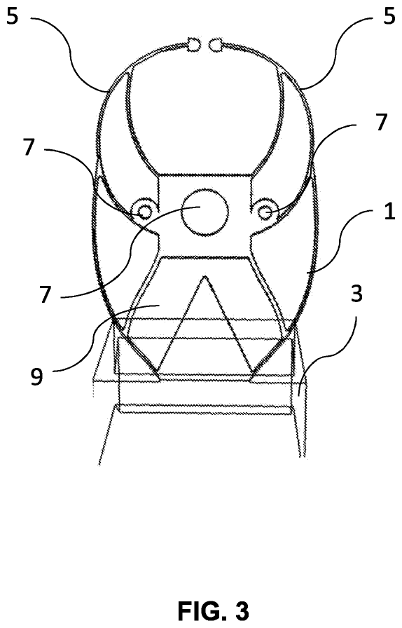

FIG. 3 shows a sample manipulator, according to yet another embodiment of the invention. The manipulator includes a partially resilient frame 1. At least two locations are provided on the frame 1 for removably positioning the manipulator. A pair of opposingly positioned arms 5 is formed at one end of the frame 1. At least one means for actuation 7 of the frame 1 is provided on the frame 1.

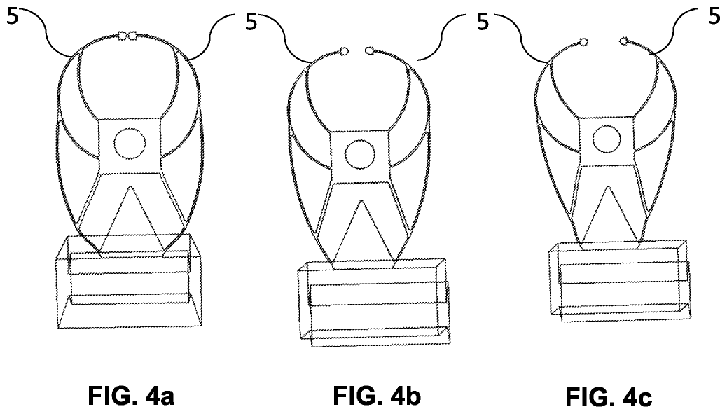

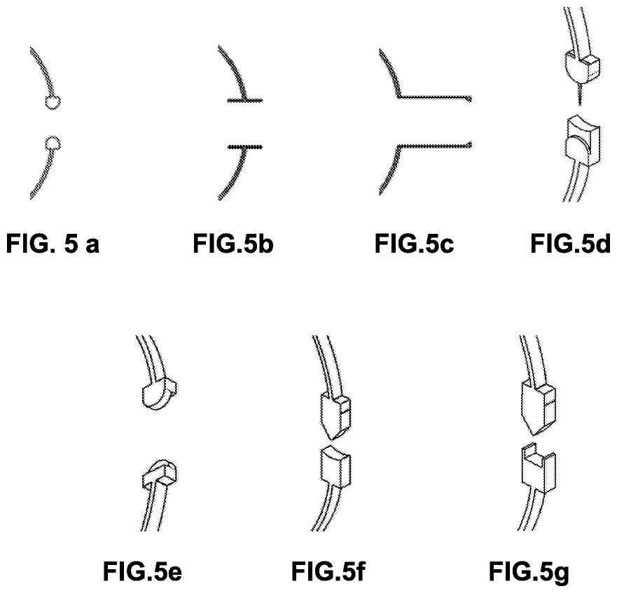

The direction of movement of the actuator determines the movement of the pair of the opposing arms 5. The movement of the arms 5 is in a plane. The plane of movement is defined with respect to the positioning of the manipulator. Further, the movement of the pair of the arms 5 is with respect to a predefined configuration of the arms 5. FIG. 4 shows different configurations of the pair of the arms of the sample manipulator, according to an embodiment of the invention. In one example of the invention, the configuration is a contact position of the pair of arms, as shown in FIG. 4(a). In another example of the invention, the configuration is a non contact position of the pair of arms, as shown in FIG. 4(b) and FIG. 4(c). Each of the arms is further provided with projections for establishing contact with the sample. The projections can be of any geometrical shape. Further, the projections on either arm can be identical or otherwise. FIG. 5(a)-5(g) shows various projections that can be configured, according to various embodiment of the invention.

Another embodiment of the invention provides a sample manipulator for out of plane manipulation. The manipulator includes a partially resilient frame having at least two locations on the frame for removably positioning the manipulator. At least one pair of opposingly positioned projections is formed at one end of the frame. A means for actuation of the frame is provided on the frame for enabling a multi-axial actuation. The frame is formed in a manner, as described earlier. The locations on the frame can be preformed, in one example. Alternatively, the locations can be identified and affixed, based on the position of the means for actuation. In one example of the invention, the two pairs of arms can be formed out of the frame. Alternatively, one pair of the arms can be formed out of the frame. The second pair of the arms can be formed separately and affixed perpendicular to the first pair of the arms. In one example of the invention, the two pairs of arms can be actuated by a single actuator. Alternatively, each of the pair can be actuated by a separate actuator. Examples of each of the actuator include but are not limited to manual screw based actuator, micrometer screw-gauge, pneumatic actuator, linear actuator, hydraulic actuator, piezo-electric actuator, piezo-resistive actuator, thermal actuator, electro-thermal actuator, electrostatic actuator, Shape memory alloys-based actuator, biohybrid actuator, muscle-based actuator, jet-based or a combination of thereof. The mode of actuation of each of the actuator can be achieved either by a mechanical based, an electrical based actuation, an electronical based actuation or a force-field based actuation. Further, the mode of actuation can be regulated either at the point of actuation or remotely. Further, each of the actuator along with the corresponding mode of actuation can either by identical or distinct. The movement of each of the pair of the arms can be synchronized with respect to the other. Alternatively, the movement of each of the pair of arms can be distinct with respect to the other.

Yet another embodiment of the invention provides a sample manipulator that includes a frame having a first end configured for removably fixing the manipulator. An intermediate region is configured to receive an operating means. A second end is configured for releasably holding the sample.

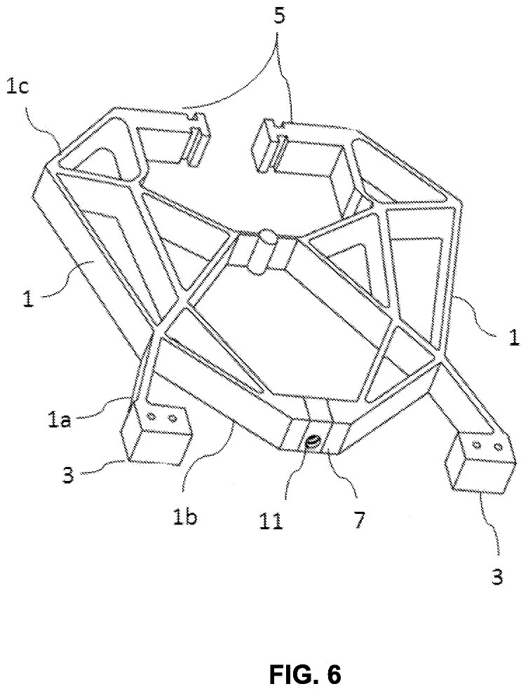

FIG. 6 shows a perspective view of the sample manipulator, according to an embodiment of the invention. The frame 1 is formed to have a first end 1a configured for removably fixing the manipulator, for example, a mount 3. An intermediate region 1b is configured to receive an operating means 7. A second end 1c is configured for releasably holding the sample. The intermediate region 1b has a threaded groove 11 to receive the operating means (not shown). The intermediate region 1b has a mount 3 provided for removably fixing the manipulator. The first end 1a has a pair of opposingly positioned arms 5 for retaining the sample. In one example of the invention, the arms 5 are further modified to house a sliding arrangement for enabling various modes of manipulation. In one example of the invention, the operating means is a screw for inducing extension of the manipulator. A mount 3 is provided for enabling ease of operation of the manipulator.

A further embodiment of the invention provides a manipulator for manipulation of a sample that includes a partially resilient frame. At least two locations are provided on the frame for removably positioning the manipulator. A pair of opposingly positioned arms is formed at one end of the frame. At least one means for actuation of the frame is provided on the frame. The frame is formed out of a material, as described herein above. The locations on the frame can be preformed, in one example.

Alternatively, the locations can be identified and affixed, based on the position of the means for actuation. In one example of the invention, the two pairs of arms can be formed out of the frame. Alternatively, the pair of the arms can be formed separately and affixed perpendicular to the first pair of the arms. In one example of the invention, the pairs of arms can be actuated by a single actuator. Alternatively, each of the arms of the pair can be actuated by a separate actuator. Examples of actuator are as described earlier herein.

Example: In one specific example of the invention, the manipulator, as disclosed herein, is fabricated using 3D printing. The fabricated manipulator has a footprint of 10 mm×10 mm with an in-plane width of 200 micrometer and an out of plane thickness of 200 micrometer. The material used for 3D printing is a commercially available biocompatible polyjet photopolymer sold under the trade name MED610. MED610 is a rigid medical rapid prototyping material, whose Young's modulus is 2 GPa and flexural strength is 75 MPa. In one specific example, the sample to be manipulated is a glass bead of diameter in the range of about 350 micrometer to about 500 micrometer. The sample is suspended in a medium on a glass slide supported by a XY stage from Prior Scientific, Inc. A micropositioner, MP-285 from Sutter, is used to get the sample between jaws of the device. The manipulators then actuated using a computer-controlled linear actuator. An actuation of 500 mm is required to grasp the sample. An FEM simulation is done to compute the stress induced in the sample due to the grasping by the manipulator. It is observed that the beams 10 of FIG. 1 have the maximum stress induced. It is observed that the stress induced is well within the permissible range of the material. The maximum stress induced on the beams is estimated to be about 520 MPa.

The manipulator, as described herein above through various embodiments is capable of manipulating samples. The dimension of the sample is in the range of about 10⁻⁶ m to about 10⁻⁹ m. Examples of manipulation include a task that includes but is not limited to squeezing, gripping, grasping, rolling, stretching, bending, piercing, probing and tearing. Examples of samples include but are not limited to single biological cells or a cluster of biological cells, bacteria, other biological microorganisms, tissues, scaffolds, tissue-scaffolds, biopsies, gametes, zygotes, organelles, platelets, sand-particles, mechanical components, crystals and crystalline structures. The manipulator, as described herein is capable of being operatively coupled to a microscope for performing manipulation of the sample.

The invention provides a sample manipulator, as described herein above through various embodiments has several advantages. One advantage is the force measurement. The actuation of the manipulator introduces a force at the point of actuation that is transmitted through the partially resilient frame to the pair of arms. The force generated can be estimated through a plurality of sensors positioned along the resilient frame for estimating the magnitude of force. Additionally, the force can also be estimated by a non-invasive sensor such as a camera. The displacements are measured using the camera and then force is computed. Another advantage of the manipulator is the multiple reuse of the mechanism. Yet another advantage of the manipulator as disclosed herein is the scalability in terms of the size of the manipulator. A further advantage of the manipulator is stiffness tuning. The mechanism described herein briefly shall be explained in detail as exemplary embodiment of the invention. Additionally, the fabrication, assembly and operation of the manipulator are simple, reliable, robust and easily reproducible. They can be actuated using simple instrumentation that also adds to their advantages.

The foregoing description of the invention has been set merely to illustrate the invention and is not intended to be limiting. Since modifications of the disclosed embodiments incorporating the spirit and substance of the invention may occur to person skilled in the art, the invention should be construed to include everything within the scope of the appended claims and equivalents thereof.

We claim:

1. A sample manipulator for in-plane manipulation comprising of a partially resilient frame (1) having
at least one position (3) for removably positioning the manipulator;
at least one pair of opposingly positioned projections (5) formed at one end of the frame (1); and
at least one means for actuation (7) of the frame (1), provided on the frame (1).

2. The manipulator of claim 1, wherein the means for actuation (7) is either a uni-directional actuator or a bi-directional actuator.

3. The manipulator of claim 1, wherein the mode of actuation induces a movement of the pair of projections (5) from a predefined configuration of the projections (5).

4. The mode of actuation of claim 3, wherein the movement is either a horizontal motion or a vertical motion.

5. The mode of actuation of claim 3, wherein the predefined configuration is either a contact position or a non contact position of the pair of projections (5).

6. A sample manipulator for out of plane manipulation comprising of a partially resilient frame (1) having
at least one position (3) on the frame (1) for removably positioning the manipulator;
at least one pair of opposingly positioned projections (5) formed at one end of the frame (1); and
at least one means for actuation (7) of the frame (1), provided on the frame (1).

7. The manipulator of claim 6, wherein the means for actuation (7) is a uni-directional actuator, a bi-directional actuator or a multi-axial actuator.

8. The manipulator of claim 6, wherein the mode of actuation induces a movement of the pair of projections (5) from a predefined configuration of the projections (5).

9. The manipulator of claim 8, wherein the movement is at least one of a horizontal motion, a vertical motion or a combination thereof.

10. (canceled)

11. The manipulator of claim 1, wherein the frame (1) is either a partially resilient frame or a flexible frame.

12-13. (canceled)

14. The manipulator of claims 1, wherein the projections (5) are of geometrical shape.

15. The manipulator of claims 1, wherein the actuation introduces a force at the point of actuation that is transmitted through the partially resilient frame (1) to the pair of projections (5).

16. The manipulator of claims 1, wherein the dimension of the sample is in the range of about 10⁻² m to about 10⁻⁹ m.

17. The manipulator of claims 1, wherein additionally a plurality of sensors can be positioned along the resilient frame for estimating the magnitude of force.

18. The manipulator of claims 1, wherein the manipulation is a task, the task selected from a list comprising of squeezing, gripping, grasping, rolling, stretching, bending, piercing, probing and tearing.

19. The manipulator of claims 1, wherein the sample is a biological sample selected from a list comprising of biological cells, tissues, scaffolds, tissue-scaffolds, biopsies, zygotes, organelles, platelets, peptides, polypeptides, and macromolecules.

20. The manipulator of claims 1, wherein the sample is selected from a list comprising of crystals, crystalline structures, amorphous compounds, wood, wood composites, polymers or combination thereof.

21. The manipulator of claim 6, wherein the frame (1) is either a partially resilient frame or a flexible frame.

22. The manipulator of claims 6, wherein the projections (5) are of geometrical shape.

23. The manipulator of claims 6, wherein the actuation introduces a force at the point of actuation that is transmitted through the partially resilient frame (1) to the pair of projections (5).

24. The manipulator of claims 6, wherein the dimension of the sample is in the range of about 10⁻² m to about 10⁻⁹ m.

25. The manipulator of claims 6, wherein additionally a plurality of sensors can be positioned along the resilient frame for estimating the magnitude of force.

26. The manipulator of claims 6, wherein the manipulation is a task, the task selected from a list comprising of squeezing, gripping, grasping, rolling, stretching, bending, piercing, probing and tearing.

27. The manipulator of claims 6, wherein the sample is a biological sample selected from a list comprising of biological cells, tissues, scaffolds, tissue-scaffolds, biopsies, zygotes, organelles, platelets, peptides, polypeptides, and macromolecules.

28. The manipulator of claims 6, wherein the sample is selected from a list comprising of crystals, crystalline structures, amorphous compounds, wood, wood composites, polymers or combination thereof.
